(19) United States
(12) Patent Application Publication
(10) Pub. No.: US 2009/0159085 A1
(43) Pub. Date: Jun. 25, 2009

(54) Piezoelectric polymer cuff for use in an artificial airway

(75) Inventor: Michael Sleva, Atlanta, GA (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 12/004,585

(22) Filed: Dec. 21, 2007

(51) Int. Cl. A61M 16/00

(52) U.S. Cl. 128/207.15

(57) ABSTRACT

A medical device for insertion into a human body is provided. The medical device includes an inflation cuff composed of piezoelectric polymers. Upon inflation of the inflation cuff, the piezoelectric polymers of the inflation cuff exhibit an electrical potential. This electrical potential is measured and correlated to an inflation cuff pressure. The pressure of the inflation cuff is then adjusted, as necessary to prevent sustained underinflation or overinflation of the inflation cuff.

### BACKGROUND

Current endotracheal tubes and other airway sealing medical devices utilize an inflation cuff that presses against the sides of the trachea or other body cavity as a fluid barrier. The integrity of this cuff seal is critical, as there is a substantial amount of secretions that accumulate on top of the tubes during use that are harmful to the patient if allowed to enter the patient's lungs or pass beyond such other devices in other applications.

Often, during a medical procedure, the medical professional will be required to manipulate the endotracheal tube in a transverse or longitudinal manner or the patient may shift which may result in movement of the tube. Due to this movement, as well as other factors, the inflation cuff may slowly deflate over time causing gaps between the inflation cuff and the tracheal walls.

These gaps are particularly disturbing because they allow for the passage or aspiration of secretions into the lungs. These secretions often contain bacteria that may cause ventilator acquired pneumonia which according to some studies accounts for 7-8% of all deaths in hospital Intensive Care Units.

In addition to deflation of cuffs, overinflation of cuffs may occasionally occur. Overinflation of inflation cuffs may cause injury to the trachea by damaging its mucous membranes.

As a solution to the problems of deflation and overinflation, high volume, low pressure inflation cuffs have been used. These inflation cuffs, in their unfolded, unexpanded state, have a diameter greater than the diameter of the cavity in which it is inflated. This maintains the filling pressure of the balloon below the blood flow pressure of the walls of the cavity the inflation cuff fills.

Although high volume, low pressure inflation cuffs are generally effective, occasionally, inflations cuff may still deflate causing secretions to travel past the cuffs into the lungs. Additionally, overinflation may still occur despite the enhanced dimensions of these cuffs.

Thus, an economical mechanism for measuring the pressure of an inflated inflation cuff, in use, is desirable to prevent sustained underinflation and/or overinflation of an inflation cuff.

### SUMMARY OF INVENTION

The present invention provides for a medical device for insertion into a human body. The medical device includes a tube having a distal end, a proximal end, and at least one inflation lumen having at least one opening. The medical device further includes at least one inflation cuff attached to the tube and situated over the opening of the inflation lumen. The inflation cuff includes an internal surface, an external surface, and a piezoelectric material. The inflation cuff further includes a first electrode that is in communication with the internal surface of the inflation cuff and the piezoelectric material of the inflation cuff. The inflation cuff also includes a second electrode that is in communication with the external surface of the inflation cuff and the piezoelectric material of the inflation cuff. Both the first and second electrodes may be composed of gold, silver, copper, conducting paint or combinations thereof and may be adhered to the internal and/or external surfaces of the inflation cuff by an adhesion layer, desirably composed of chrome. Additionally, the medical device also includes a means for monitoring an amount of electrical potential developed between the first electrode and the second electrode across the piezoelectric material.

Desirably, the means for monitoring the amount of electrical potential developed between the first electrode and the second electrode across the piezoelectric material is a first lead wire in communication with the first electrode and second lead wire in communication with the second electrode. Desirably, the first lead wire and second lead wire may be composed of copper, silver, gold or aluminum and are adapted to measure electrical potential developed between the first electrode and second electrode respectively. This electrical potential may be measured by connecting the first and second lead wires to an analog or digital monitoring device outside the patient's body which is adapted for measuring electrical potential in terms of voltage.

The piezoelectric material of the invention desirably is poly(vinylidene fluoride) or poly(vinylidene fluoride-trifluoroethylene). These materials may compose the internal and external surfaces of the inflation cuff. Additionally, the inflation cuff of the invention may also include one or more non-piezoelectric materials. These non-piezoelectric materials may be disposed between the internal surface and external surface of the inflation cuff.

A further aspect of the invention includes an inflation cuff adapted for use with a medical device. The inflation cuff includes an internal surface, an external surface, and a piezoelectric material. The inflation cuff further includes a first electrode that is in communication with the internal surface of the inflation cuff and the piezoelectric material of the inflation cuff. The inflation cuff also includes a second electrode that is in communication with the external surface of the inflation cuff and the piezoelectric material of the inflation cuff. Additionally, the medical device also includes a means for monitoring an amount of electrical potential developed between the first electrode and the second electrode across the piezoelectric material.

Yet another aspect of the invention includes a system for measuring the interface pressure between the inflation cuff and the tissue of a body cavity. The system includes an inflation cuff having an internal surface, an external surface, and a piezoelectric material. The inflation cuff further includes a first electrode that is in communication with the internal surface of the inflation cuff and the piezoelectric material of the inflation cuff. The inflation cuff also includes a second electrode that is in communication with the external surface of the inflation cuff and the piezoelectric material of the inflation cuff. Additionally, the medical device also includes a means for monitoring an amount of electrical potential developed between the first electrode and the second electrode across the piezoelectric material. The system further includes a means for correlating an amount of electrical potential developed between the first electrode and the second electrode across the piezoelectric material to an amount of interface pressure between the inflation cuff and the tissue of a body cavity.

### BRIEF DESCRIPTION OF DRAWINGS

FIG. 1A is a perspective view of medical device having the inflation cuff of the present invention.

FIG. 1B is a sectional view of the piezoelectric layer of the inflation cuff.

FIG. 2. is a sectional view of an inflation cuff having a non-piezoelectric layer surrounded by piezoelectric layers.

### DETAILED DESCRIPTION

The medical device of the present invention provides for inflation cuff composed of a piezoelectric polymer. The piezoelectric polymer, when stressed, through inflation for example, creates an electrical potential across its surface. This allows electricity to be measured and correlated to an inflation pressure. Thereafter, the pressure of the inflation cuff may be adjusted to prevent sustained underinflation and subsequent leaking of secretions past the inflation cuff and to prevent sustained overinflation and subsequent damage to the mucosal lining of the trachea. Although the medical devices are described herein as endotracheal or other airway devices, any medical device utilizing an inflation cuff may utilize the piezoelectric polymers disclosed herein.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

In the interests of brevity and conciseness, any ranges of values set forth in this specification contemplate all values within the range and are to be construed as support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 14; 1-3; 1-2; 2-5; 2-4; 2-3; 3-5; 3-4; and 4-5.

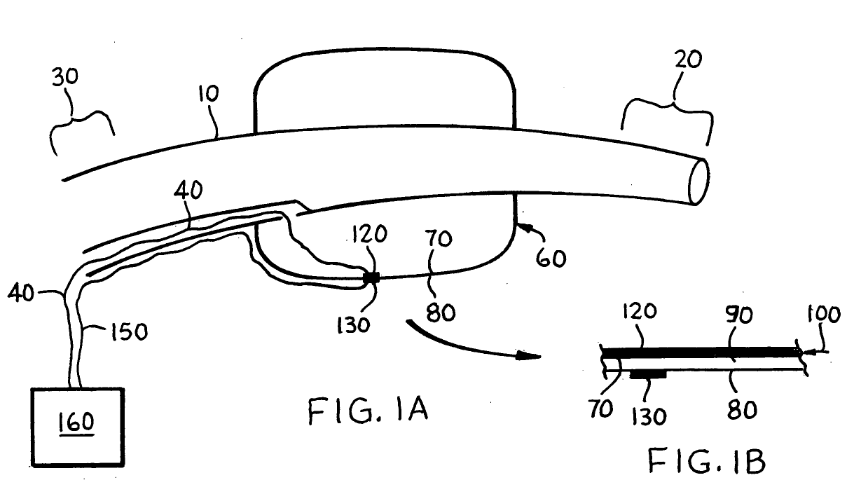

Referring to FIGS. 1A and 1B, a medical device for insertion into a human body cavity is provided. The medical device includes an inflation cuff 60 attached to a tube 10, desirably an endotracheal tube. The tube has a distal end 20, a proximal end 30, and at least one inflation lumen 40 having at least one opening for introduction of fluids into the inflation cuff 60, the opening being connected to the inflation lumen. These fluids include gases and liquids.

The medical device, desirably an endotracheal tube, may include any flexible material which is compatible with the elongate body cavity into which it is inserted and which does not cause irritation of the cavity. These materials include, but are not limited to, latex, silicone, polyvinyl chloride, polyurethane or polytetrafluoroethylene.

The tube may be manufactured by any method known in the art of manufacturing tubing. A non-limiting example of an endotracheal manufacturing process suitable for this purpose is the coextrusion of two tubes (coextrusion being a process known and understood by those having skill in the manufacture of tubing), an outer tube and an inner tube. Additional, non-limiting examples include utilizing reinforcement inserts such as wire inside the shaft of the endotracheal tube, utilizing a spiral wound reinforcement, or utilizing a stabilizing mesh incorporated into the wall of the endotracheal tube.

Referring again to FIGS. 1A and 1B, the apparatus also include an inflation cuff 60 attached to the body of the tube 10. The inflation cuff may be attached to the tube utilizing gluing, welding, suturing or any other known method in the art for attaching an inflation cuff to an endotracheal or other elongate tube. The inflation cuff may be desirably preformed or may be alternatively formed by methods such as blow-molding, dip coating, or spin coating.

When the inflation cuff is preformed it is preformed to exhibit dimensions greater than the diameter of the elongate body cavity in which it is to be inserted, for example, the trachea. In this regard, it is preformed to exhibit dimensions from between about 1 to about 20 percent, desirably about 1 to about 10 percent, and more desirably between about 3 percent to about 7 percent beyond the diameter of the trachea. It is also contemplated that the preformed inflation cuff may exhibit dimension greater than 20 percent beyond the diameter of the trachea. While these dimensions are variable, they may be readily determined by one of ordinary skill in the art utilizing conventional techniques.

As an alternative to performing, the inflation cuff may be constructed by any of the other various techniques well-known to those skilled in the art. For example without limitation, the polymer can be dip-coated on a mandrel that has a defined size and shape, desirably greater than the diameter of the tracheal cavity. When removed from the mandrel, the inflation cuffs in their uninflated or collapsed state will assume two dimensions (length and width) of the mandrel without incurring any tensional force in the polymer.

The inflation cuff may also be formed by spin-coating in a hollow mold. When the mold is removed, as in the case of a dip-coated mandrel, the inflation cuff will assume dimensions that are the same as the interior dimensions of the hollow mold.

In addition, inflation cuffs may be formed by injection or blow molding. In this process, a pre-formed length of tubing made of the polymer is placed in a hollow mold having internal dimensions that reflect the desired dimensions of the inflation cuff to be formed. One end of the tube is sealed off and a working fluid is injected into the open end of the tube with sufficient force to cause the working fluid to expand the tubing until the wall of the tubing is in intimate contact with the inner surface of the mold. The polymer is then annealed, if desired, and cooled after which the mold is removed leaving a portion of the tubing as an inflation cuff.

The above are but a few methods of forming the inflation cuff. Others will be apparent to those skilled in the art. All such methods are within the scope of this invention.

Various materials may be used to form the inflation cuffs. These materials include, but are not limited to, piezoelectric polymers, for example poly(vinylidine fluoride) (PVDF) or poly(vinylidene fluoride-trifluoroethylene) P(VDF-TrFE). Other materials would also be suitable so long as they are adapted to exhibit piezoelectric properties and are able to be processed into inflation cuffs having walls, desirably microthin walls on the order of about 5 to about 100 micrometers, more desirably into a range of between about 5 to about 50 micrometers, yet even more desirably between about 5 and about 20 micrometers, and yet even more desirably between about 5 and about 15 micrometers. It is also contemplated that the walls may have a thickness of less than about 5 micrometers. Additionally, although the thickness of the microthin walls may vary, it is desirable that the thickness of the microthin material remain consistent throughout the inflation cuff.

The thickness of the walls of the inflation cuff may be measured by conventional measuring techniques. These techniques include the use of LITEMATIC which is a low-contact-force (0.01 N) thickness measuring device (LITEMATIC VL-50 available from KABUSHIKI KAISHA MITSUTOYO, Japan).

It is also desirable, that upon inflation of the microthin inflation cuff, the inflation cuff will exhibit outward pressure towards the walls of the body cavity in embodiments where the inflation cuff is used in the trachea. Desirably, upon inflation, pressure inside the inflation cuff will be 30 millibars or less, desirably from about 10 to about 30 millibars, and an equal amount of pressure will be exerted upon the tracheal wall. It is also contemplated that the pressure could be less than 10 millibars for short periods of time during movement of the tube or during part of the breathing cycle.

These microthin walls are desirable because, as mentioned above, the diameter of the inflation cuff is greater than the diameter of the trachea. This allows the inflation cuff, upon inflation in the trachea to inhibit and/or arrest the free flow of secretions into the lungs. A detailed discussion of this phenomenon is found in U.S. Pat. No. 6,526,977 and U.S. Pat. No. 6,802,317 both of which are incorporated herein by reference in their entireties.

The piezoelectric polymers of the present invention exhibit a variety of electrical and mechanical properties such as piezoelectricity. Piezoelectricity is the ability of a material, for example PVDF and P(VDF-TrFE), to convert a mechanical stimulus to an electrical response, and vice versa. This characteristic enables PVDF and P(VDF-TrFe) to act as a sensor. In this regard, when a mechanical stress is applied to a piezoelectric material, the piezoelectric material will produce an electrical potential 100 across its boundaries. This electrical potential value may be measured and correlated to a mechanical stressor value, i.e. pressure value, that can be easily calculated by one of ordinary skill in the art by use of an algorithm or some other method of calculation. This is due to the fact that electrical potential and voltage is proportional to the amount of pressure exerted against the internal and external surface of the piezoelectric material, for example, the internal 70 and external 80 surfaces of the inflation cuff.

The source of the piezoelectric characteristics of the polymers of the present invention is a dipole created by the Fluorine and Carbon elements bonded in their structures. A dipole is a pair of electric charges (or magnetic poles) separated by a small distance and having equal magnitude and opposite polarity.

In these polymers, the dipoles are generally randomly oriented, thus little or no polarization of these materials will occur. This is called the alpha phase. In order to enter the beta phase (where the polymeric materials will be useful as piezoelectrics), the polymers must be subjected to a large electric field, typically at elevated temperature, through use of an electrostatic generator or similar device as known in the art, so that the dipoles will align in the direction of the field. When the electric field is removed, the dipoles will remain substantially aligned in the direction of the field. Thereafter when the dipoles are physically stressed and their lengths altered, the polarization of the material also changes. It is this alignment of the dipoles that causes the polymeric material to be useful as a piezoelectric.

Returning to FIGS. 1A and 1B, after the piezoelectric material 90 of the inflation cuff has entered the beta phase, a first electrode 120 is applied to the internal surface 70 of the inflation cuff and a second electrode 130 is applied to the external surface 80 of the inflation cuff. The electrodes may be composed of, for example gold, silver, copper, conductive paint, combinations thereof, or any other conductive metal capable of developing electrical potential between electrodes. Additionally, the electrodes may be adhered to the internal and/or external surfaces of the inflation cuff by use of an adhesion layer composed of chrome or any other conductive metal capable of adhering to piezoelectric material to form an adhesion layer with the piezoelectric material. The adhesion layer is generally between about 10 and about 100,000 angstroms thick.

After inflation of the cuff, a stress, for example pressure, applied to the piezoelectric material results in electrical potential generated across the piezoelectric material. The electrodes in communication with the piezoelectric material “read” this electric potential by electrical connection to a monitoring device 160 outside the patient's body. In this regard, a first electrical lead 140 and a second electrical lead 150 made of copper, silver, gold, aluminum, or another conductive metal connected to the first 120 and second 130 electrodes, respectively, inside the patient's body cavity harness and transmit electrical potential safely through the patient's body cavity to the outside analog or digital monitoring device, i.e an oscilloscope, for measuring and displaying electrical potential. This voltage value is then used to calculate the corresponding inflation pressure associated with the inflation cuff. Depending upon the calculated value of the inflation pressure, the inflation cuff may be inflated or deflated as needed, manually or through a suitable automated inflation mechanism.

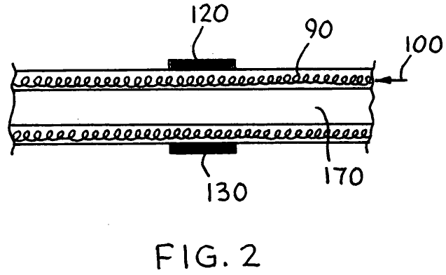

Referring to FIG. 2, in an alternative embodiment, the piezoelectric materials of the present invention may be laminated to an inflation cuff composed of non-piezoelectric 170 materials. In this regard, after the piezoelectric materials enter the beta phase, one or more layers of piezoelectric polymers 90 may be laminated to the external surface of the non-piezoelectric material and one or more layers of piezoelectric material may be laminated to the internal surface of the non-piezoelectric material. As discussed above, electrodes may then be applied to the internal and external surface of the piezoelectric material and electrical potential may then be measured in order to determine the pressure within the inflation cuff and the pressure between the walls of the body cavity and external surface of the inflation cuff. Additionally, it may also be desirable for multiple electrodes, having corresponding lead wires, to be in communication with both the internal and external surfaces of the inflation cuff to measure electrical potential generated at various locations on the inflation cuff to ensure uniform inflation of the inflation cuff. Alternatively, it is also possible for a layer of the inflation cuff to include both piezoelectric and non-piezoelectric materials.

Non-piezoelectric material suitable for formation of inflation cuffs for use with the present invention include, but are not limited to, polyurethane (PU), low-density polyethylene (LDPE), polyvinyl chloride (PVC), polyamide (PA) or polyethylene teraphthalate (PETP). Additionally, copolymer admixtures for modifying the characteristics of the material may be used, for example a low density polyethylene and ethylene-vinylacetate copolymer (LDPE-EVA), or blends of the above mentioned materials (e.g. PU with PVC or PU with PA) would be considered suitable for forming the inflation cuff. Other materials would also be suitable so long as they do not interfere with the transmission of electrical potential across the piezoelectric layers.

In addition to the medical device and inflation cuff described above, a system for measuring the interface pressure is provided. The system includes an inflation cuff having an internal surface, an external surface, and a piezoelectric material. The inflation cuff further includes a first electrode that is in communication with the internal surface of the inflation cuff and the piezoelectric material of the inflation cuff. The inflation cuff also includes a second electrode that is in communication with the external surface of the inflation cuff and the piezoelectric material of the inflation cuff. Additionally, the medical device also includes a means for monitoring an amount of electrical potential developed between the first electrode and the second electrode across the piezoelectric material. This means for monitoring is lead wires attached to the opposing electrodes. These lead wires harness the electrical potential developed across the piezoelectric material and electrically communicates it to a monitor outside the body of the patient. The monitor, for example, an oscilloscope displays the electrical potential as a voltage value and the voltage is correlated to a cuff inflation pressure by utilizing a mathematical algorithm, or some other mechanism known in the art, to convert voltage (which is directly proportional to inflation pressure) to an inflation pressure value so that the inflation pressure may be adjusted, if necessary.

I claim:

1. A medical device for insertion into a human body, the medical device comprising:
a tube comprising a distal end, a proximal end, and at least one inflation lumen having at least one opening;
at least one inflation cuff attached to the tube, the at least one inflation cuff comprising an internal surface, an external surface, and a piezoelectric material, the at least one inflation cuff further comprising a first electrode in communication with the internal surface of the inflation cuff and in communication with the piezoelectric material of the inflation cuff, and a second electrode in communication with the external surface of the inflation cuff and in communication with the piezoelectric material of the inflation cuff, wherein the at least one inflation cuff is situated over the at least one opening of the inflation lumen; and
a means for monitoring an amount of electrical potential developed between the first electrode and the second electrode across the piezoelectric material.

2. The medical device of claim 1, wherein the means for monitoring the amount of electrical potential developed between the first electrode and the second electrode across the piezoelectric material comprises a first lead wire in communication with the first electrode and second lead wire in communication with the second electrode, the first lead wire and second lead wire being adapted to measure electrical potential developed between the first electrode and second electrode respectively, and further being adapted to pass to the outside environment.

3. The medical device of claim 2, wherein the first lead wire and second lead wire comprises copper, silver, or aluminum.

4. The medical device of claim 2 wherein the means for monitoring the amount of electrical potential developed from the first electrode to the second electrode across the piezoelectric material further comprises the connection of a first lead wire and second lead wire with an analog or digital monitoring device outside of a patient's body, the monitoring device being adapted for measuring electrical potential in terms of volts.

5. The medical device of claim 1, further comprising at least one adhesion layer on the external surface of the inflation cuff and at least one adhesion layer on the internal surface of the inflation cuff for adhering the first electrode and second electrode to the internal surface of the inflation cuff and external surface of the inflation cuff, respectively.

6. The medical device of claim 5, wherein the at least one adhesion layer comprises chrome.

7. The medical device of claim 6, wherein the first electrode and the second electrode comprise gold, silver, copper, conducting paint, or combinations thereof.

8. The medical device of claim 1, wherein the piezoelectric material is poly(vinylidene fluoride) or poly(vinylidene fluoride-trifluoroethylene).

9. The medical device of claim 1, wherein the at least one inflation cuff further comprises a non-piezoelectric material.

10. The medical device of claim 9, wherein the piezoelectric material is in communication with non-piezoelectric material, the piezoelectric material comprises the internal surface and external surface of the inflation cuff, and the non-piezoelectric material is disposed between the internal surface and external surface of the inflation cuff.

11. An inflation cuff adapted for use with a medical device, the inflation cuff comprising:
an internal surface, an external surface, and a piezoelectric material, the at least one inflation cuff further comprising a first electrode in communication with the internal surface of the inflation cuff and in communication with the piezoelectric material of the inflation cuff, and a second electrode in communication with the external surface of the inflation cuff and in communication with the piezoelectric material of the inflation cuff; and
a means for monitoring an amount of electrical potential developed between the first electrode and the second electrode across the piezoelectric material

12. The medical device of claim 11, wherein the means for monitoring the amount of electrical potential developed between the first electrode and the second electrode across the piezoelectric material comprises a first lead wire in communication with the first electrode and second lead wire in communication with the second electrode, the first lead wire and second lead wire being adapted to measure electrical potential developed between the first electrode and second electrode respectively.

13. The medical device of claim 12, wherein the first lead wire and second lead wire comprises copper, silver, or aluminum.

14. The medical device of claim 12 wherein the means for monitoring the amount of electrical potential developed from the first electrode to the second electrode across the piezoelectric material further comprises the connection of a first lead wire and second lead wire with an analog or digital monitoring device outside of a patient's body, the monitoring device being adapted for measuring electrical potential in terms of volts.

15. The medical device of claim 11, further comprising at least one adhesion layer on the external surface of the inflation cuff and at least one adhesion layer on the internal surface of the inflation cuff for adhering the first electrode and second electrode to the internal surface of the inflation cuff and external surface of the inflation cuff, respectively.

16. The medical device of claim 15, wherein the at least one adhesion layer comprises chrome,

17. The medical device of claim 16, wherein the first electrode and the second electrode comprise gold, silver, copper, conducting paint, or combinations thereof.

18. The medical device of claim 11, wherein the piezoelectric material is poly(vinylidene fluoride) or poly(vinylidene fluoride-trifluoroethylene).

19. The medical device of claim 11, wherein the at least one inflation cuff further comprises a non-piezoelectric material.

20. The medical device of claim 19, wherein the piezoelectric material is in communication with non-piezoelectric material, the piezoelectric material comprises the internal surface and external surface of the inflation cuff, and the non-piezoelectric material is disposed between the internal surface and external surface of the inflation cuff.

21. The medical device of claim 11, further comprising a plurality of electrodes in communication with the external surface of the inflation cuff and a plurality of electrodes in communication with the internal surface of the inflation cuff, and a means for monitoring the amount of electrical potential generated between each of the plurality of electrodes on the external surface of the inflation cuff and a corresponding electrode located on the internal surface of the inflation cuff.

22. A system for measuring the interface pressure between an inflation cuff and the tissue of a body cavity, the system comprising:
At least one inflation cuff comprising an internal surface, an external surface, and a piezoelectric material, the at least one inflation cuff further comprising a first electrode in communication with the internal surface of the inflation cuff and in communication with the piezoelectric material of the inflation cuff, and a second electrode in communication with the external surface of the inflation cuff and in communication with the piezoelectric material of the inflation cuff;
a means for monitoring an amount of electrical potential developed between the first electrode and the second electrode across the piezoelectric material; and
a means for correlating an amount of electrical potential developed between the first electrode and the second electrode across the piezoelectric material to an amount of interface pressure between the inflation cuff and the tissue of a body cavity.
